(19) United States
(12) Patent Application Publication
(10) Pub. No.: US 2013/0144173 A1
(43) Pub. Date: Jun. 6, 2013

(54) ULTRASONIC PROBE DEVICE AND A DETECTION METHOD THEREOF

(71) Applicant: GE Medical Systems Global Technology Company, Waukesha, WI (US)

(72) Inventors: Peng HOU, Nangsu (CN); Xinchao WANG, Jiangsu (CN); Qiyuan WANG, Jiangsu (CN); Caifeng TANG, Jiangsu (CN); Haitao ZHANG, Jiangsu (CN)

(73) Assignee: GE MEDICAL SYSTEMS GLOBAL TECHNOLOGY COMPANY, LLC, Waukesha, WI (US)

(21) Appl. No.: 13/625,926

(22) Filed: Sep. 25, 2012

(30) Foreign Application Priority Data
Sep. 30, 2011 (CN) 201110312476.5

(51) Int. Cl. A61B 8/00

(52) U.S. Cl. CPC A61B 8/4483
USPC 600/459

(57) ABSTRACT

An ultrasonic probe device comprising a fixing mechanism, an ultrasonic transducer, and a rotatable mechanism, wherein the fixing mechanism and the ultrasonic transducer are connected through the rotatable mechanism, and wherein the ultrasonic transducer is rotatable on the fixing mechanism to a predetermined position for scanning.

### BACKGROUND OF THE INVENTION

Embodiments of the present invention relate to the field of ultrasonic detection and, more particularly, to an ultrasonic probe device and a detection method thereof

Ultrasonic detection is a commonly used detection method in modern medical treatment.

Existing ultrasonic medical devices usually consist of a console and a plurality of probes for different medical uses. Due to the different uses, the probes also vary in their shape, performance, etc.

A conventional probe usually consists of a cable assembly, an ultrasonic transducer, and a fixing means (a handle, for example). Because the ultrasonic transducer needs to contact the part of human body to be detected, its shape should vary with the actual detection requirements. However, in traditional probes, the ultrasonic transducers are fixedly connected to the fixing means and the ultrasonic transducers have a uniform shape. Accordingly, they have the disadvantages that the user has to endure high costs to buy different probes to match a given ultrasonic host, and that, in use, a great deal of time is required to mount and dismount different probes to meet different needs.

Therefore, there is a need for an ultrasonic probe device that is capable of achieving a flexible switch between different probes, thereby improving the diagnosing efficiency and reducing the use-cost.

### BRIEF SUMMARY OF THE INVENTION

According to an embodiment of the present invention, there is provided an ultrasonic probe device. The ultrasonic probe device comprises a fixing mechanism, an ultrasonic transducer, and a rotatable mechanism, wherein the fixing mechanism and the ultrasonic transducer are connected through the rotatable mechanism, and wherein the ultrasonic transducer is rotatable on the fixing mechanism to a predetermined position for scanning.

According to an embodiment of the present invention, there is provided a detection method using an ultrasonic probe device. The method comprises connecting an ultrasonic transducer to a fixing mechanism through a rotatable mechanism so that the ultrasonic transducer is rotatable on the fixing mechanism; and rotating the ultrasonic transducer to a predetermined position for scanning.

### BRIEF DESCRIPTION OF THE DRAWINGS

These and other features, aspects and advantage of embodiments of the present invention can be better understood when the following detailed description are read with reference to the accompanying drawings, in which:

FIG. 1 is a flow chart showing a detection method of an ultrasonic probe device according to an embodiment of the present invention;

FIG. 2 is a structural diagram of an ultrasonic probe device according to an embodiment of the present invention;

FIG. 3 is a schematic diagram showing a cable of the ultrasonic probe device according to an embodiment of the present invention extending through a rotatable mechanism to connect a handle and ultrasonic transducers of different uses;

FIG. 4 is a schematic diagram showing an end of an ultrasonic detector of the ultrasonic probe device according to an embodiment of the present invention rotated outside the handle and secured in place for scanning;

FIG. 5 is a schematic diagram showing the ultrasonic probe device according to an embodiment of the present invention being rotated for different uses;

FIG. 6 is a exemplary schematic diagram showing an ultrasonic transducer according to an embodiment of the present invention being rotated to a predetermined position and locked in said position for scanning; and

FIG. 7 is an exemplary diagram showing the rotation of an ultrasonic transducer according to an embodiment of the present invention.

### DETAILED DESCRIPTION OF THE INVENTION

Embodiments of the present invention provide an ultrasonic probe device and a detection method capable of solving the technical problems of complicated operation and high use-cost in existing ultrasonic diagnosing processes.

An embodiment of the present invention provides an ultrasonic probe device comprising a fixing means, a probe, and a rotatable mechanism, wherein the probe and the fixing means are connected via the rotatable mechanism, and wherein the probe can rotate to a predetermined position on the fixing means for scanning.

Additionally, said device can further comprise a detection switch or a position sensor to detect the position change of ultrasonic transducers of different uses and cause the related ultrasonic transducer(s) to work.

Additionally, said device can further comprise a locking rod to lock the ultrasonic transducer at a predetermined position when the locking rod drops and fits into a recess under the action of a spring.

An embodiment of the present invention provides a detection method of the ultrasonic probe device, the method comprising connecting an ultrasonic transducer to a fixing means through a rotatable mechanism so that the ultrasonic transducer can rotate on the fixing means, and rotating the ultrasonic transducer to a predetermined position for scanning.

Embodiments of the present invention will now be further described with reference to the drawings.

The ultrasonic probe device according to an embodiment of the present invention can comprise a fixing means (for example, a handle), at least two ultrasonic transducers of different shapes assembled together, a rotatable mechanism, and a cable assembly. The cable assembly consists of a cable and a connector. A flexible cable (as distinguished from the aforementioned cable) extends through the rotatable mechanism to connect the fixing means and the ultrasonic transducers. Said fixing means and said ultrasonic transducers of different shapes are connected via the rotatable mechanism, and the ultrasonic transducers can rotate to a predetermined position on the fixing means for scanning.

Said ultrasonic transducers of different shapes are ultrasonic transducers of different uses, and depending on the requirements, there can be a plurality of ultrasonic transducers of different uses. The ultrasonic transducer can include a plurality of transducer units and the shape of the transducer units can be the same or different.

The ultrasonic transducer and the fixing means are connected through the rotatable mechanism so that the ultrasonic transducer can rotate on the fixing means.

The ultrasonic probe device further comprises a detection switch or a position sensor for detecting the position change of the ultrasonic transducers of different uses and causing the related ultrasonic transducer(s) to work.

The detection switch or position sensor is disposed inside the ultrasonic transducer and the fixing means. When an ultrasonic transducer rotates around an axis within the fixing means to a particular position, the sensor inside the ultrasonic transducer and a corresponding position detection inside the fixing means will detect such change and generate a detection signal to be transmitted to a CPU so that the host can recognize the ultrasonic transducer and allow the corresponding ultrasonic transducer to work.

The cable used is a flexible cable. The flexible cable connects the fixing means and the ultrasonic transducer through a center of the rotatable mechanism, so that the cable will not wind up when the rotatable mechanism rotates.

When the ultrasonic transducer rotates to a predetermined position, it can be locked in said position for scanning (locking is achieved through a locking rod dropping and fitting into a recess under the action of a spring). Once the probe is locked in place, the detection switch will not detect the position change of the ultrasonic transducer and therefore misoperation is avoided.

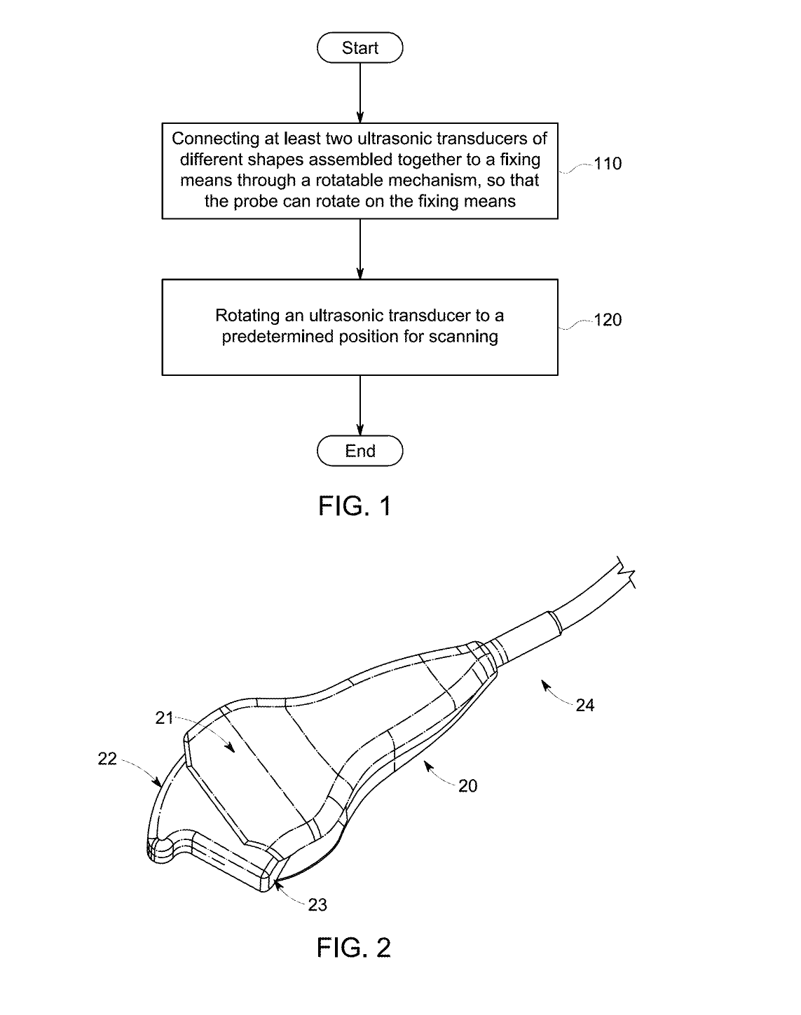

As shown in FIG. 1, the detection method of an ultrasonic probe device according to an embodiment of the present invention comprises connecting at least two ultrasonic transducers of different shapes assembled together to a fixing means through a rotatable mechanism, so that the probe can rotate on the fixing means 110; and rotating an ultrasonic transducer to a predetermined position for scanning 120.

Said ultrasonic transducers of different shapes are ultrasonic transducers of different uses, and depending on the needs, a plurality of ultrasonic transducers of different uses can be mounted. The ultrasonic transducer can be formed of a combination of a plurality of independent ultrasonic transducers each having a different shape. According to an embodiment of the present invention, an ultrasonic transducer that is formed of a combination of a plurality of independent ultrasonic transducers each having a different shape is a multifunctional ultrasonic transducer, and is generally referred to as an ultrasonic transducer.

The method may further comprise providing a cable assembly consisting of a cable and a connector, wherein the cable extends through the rotatable mechanism to connect the fixing means and the ultrasonic transducer, and wherein the cable is a flexible cable which connects the fixing means and the ultrasonic transducer through a center of the rotatable mechanism, so that the cable will not wind up when the rotatable mechanism rotates.

Said rotating an ultrasonic transducer to a predetermined position for scanning comprises determining whether an ultrasonic transducer has rotated to the predetermined position, locking the ultrasonic transducer at the predetermined position upon determining that the ultrasonic transducer has rotated to the predetermined position, and using said ultrasonic transducer for scanning.

FIG. 2 shows an ultrasonic probe device comprising a fixing means 20 (which can be a handle), a cable assembly 24 consisting of a cable and a connector, and two ultrasonic transducers (a first ultrasonic transducer 22 and a second ultrasonic transducer 23) assembled together for meeting different needs, wherein the first ultrasonic transducer 22 and the second ultrasonic transducer 23 can be integrated on the rotatable mechanism 21.

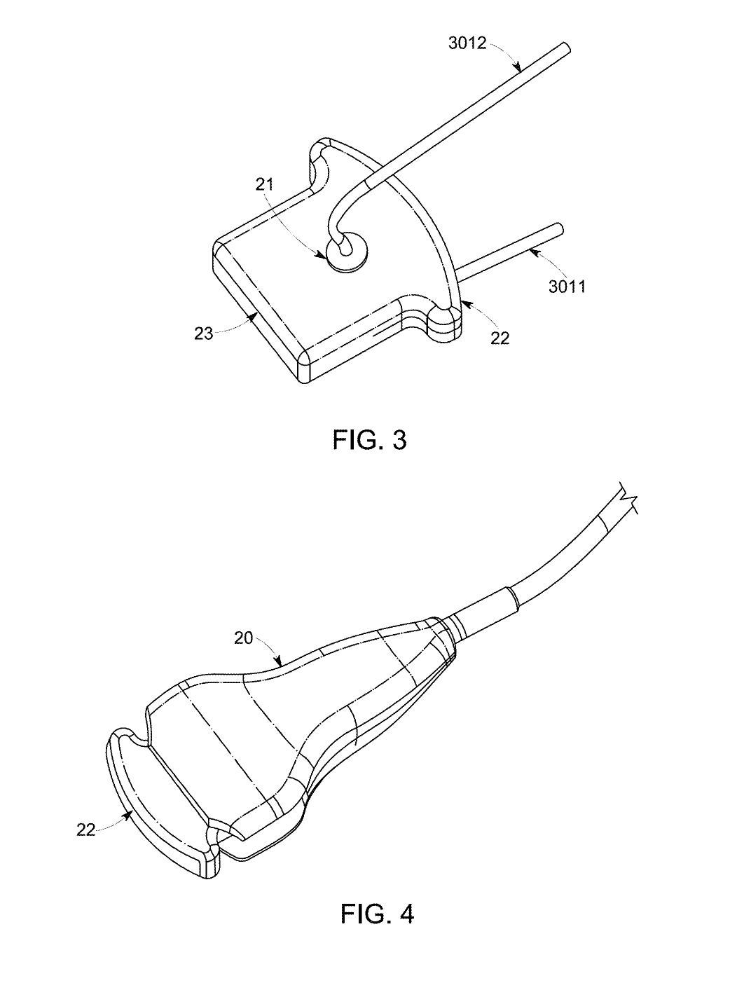

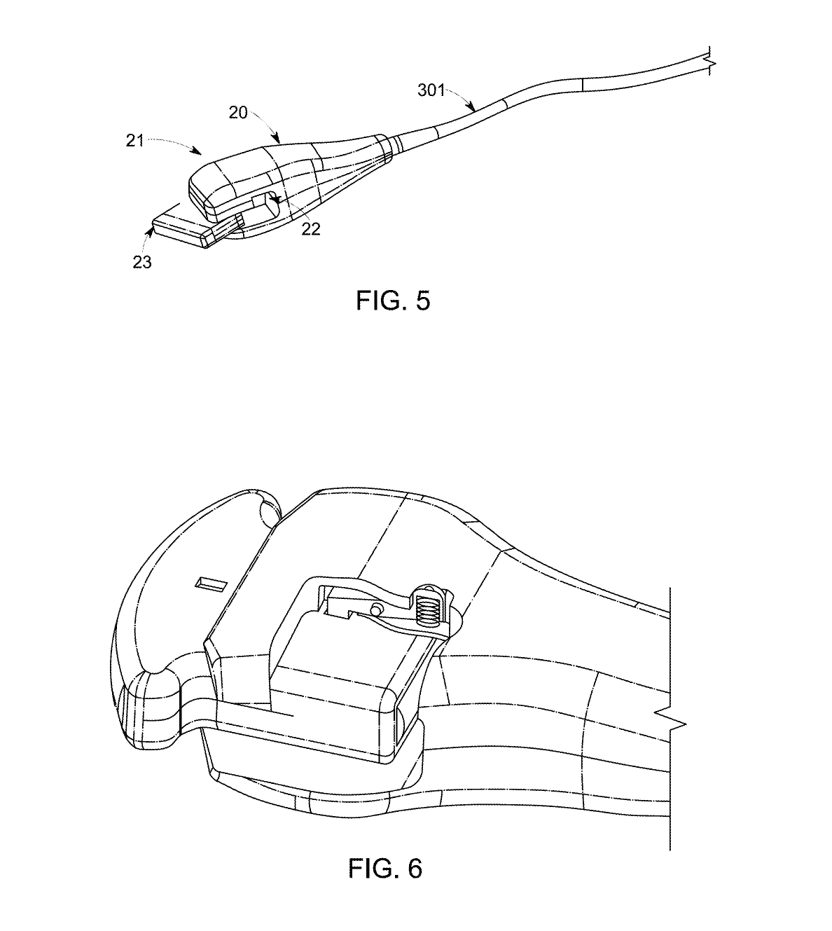

As shown in FIG. 3, a cable 301 (shown in FIG. 5) extends through the rotatable mechanism 21 to connect the fixing means 20 and the ultrasonic transducers 22, 23 of different uses, wherein the first ultrasonic transducer 22 is connected to a flexible cable 3011 and the second ultrasonic transducer 23 is connected to a flexible cable 3012.

In FIG. 4, an end of the first ultrasonic transducer 22 is rotated outside the fixing means 20 and secured in place for scanning.

FIG. 5 shows an ultrasonic probe device being rotated for a particular use. The second ultrasonic transducer 23 is connected to the fixing means 20 through the rotatable mechanism 21 so that the ultrasonic transducer 23 can rotate on the fixing means 20. The fixing means 20 is connected to cable 301.

FIG. 6 is an exemplary schematic diagram showing an ultrasonic transducer rotated to a predetermined position and locked in said position for scanning according to an embodiment of the present invention. As shown, each ultrasonic transducer has a corresponding recess. When the ultrasonic probe device rotates to a suitable angle for scanning, the locking rod will drop and fit into the corresponding recess under the action of a spring to achieve locking.

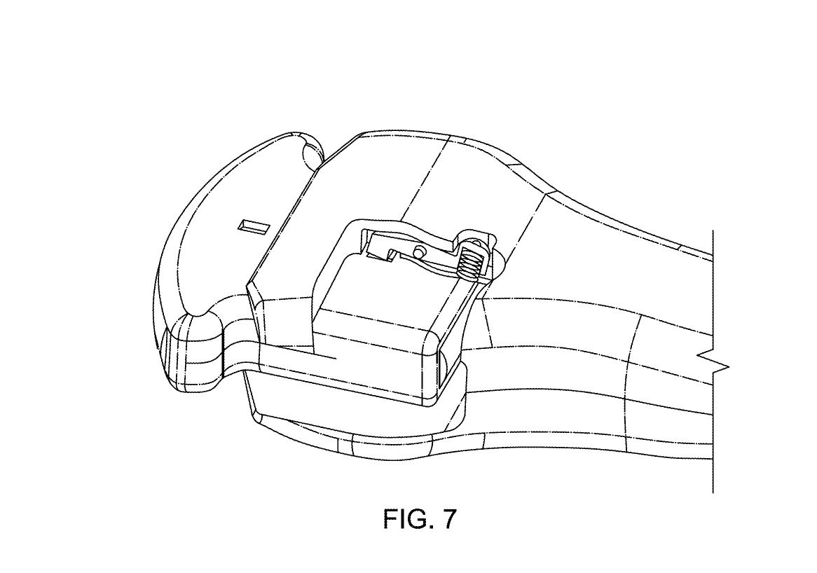

As shown in FIG. 7, when it is necessary to rotate the ultrasonic probe device, the user can press a button, as shown, with the thumb to cause the locking rod to be lifted and disengaged from the recess so that the ultrasonic probe device can be rotated.

Embodiments of the present invention can save at least 25% of the use-costs associated with conventional probes. The user only need to buy one instead of two or more probes. as is shown below:

N×probe+N×cable assembly=>N×probe+1 cable assembly (N>=2).

Thus, embodiments of the present invention eliminate the need to buy extra probes and cable assemblies and. accordingly, significantly reduce use-costs.

Embodiments of the present invention also eliminate the need for frequent mounting and dismounting of different probes for the ultrasonic host. Instead, the user can simply rotate the desired ultrasonic transducer(s) to particular position(s) for use. This saves time and simplifies operation.

As compared with the existing technology, embodiments of the present invention make it possible for the ultrasonic transducer 22, 23 to rotate on the fixing means 20 by connecting the ultrasonic transducer 22, 23 and the fixing means 20 via a rotatable mechanism 21. This simplifies the operation required in existing ultrasonic diagnosing processes, saves operation time, and reduces the use-cost of the device.

The foregoing description describes some embodiments of the present invention. However, the protection scope of the present invention is not intended to be limited thereto. Any changes or modifications that are conceivable to those skilled in the art based on the above disclosure shall be within the scope of protection of the present invention. Therefore, the protection scope of the present invention should be determined as defined by the claims.

What is claimed is:

1. An ultrasonic probe device comprising:
a fixing mechanism;
an ultrasonic transducer; and
a rotatable mechanism,
wherein the fixing mechanism and the ultrasonic transducer are connected through the rotatable mechanism, and wherein the ultrasonic transducer is rotatable on the fixing mechanism to a predetermined position for scanning.

2. The device according to claim 1, further comprising a cable assembly, wherein the cable assembly comprises a cable and a connector configured to connect an ultrasonic host, and wherein the cable extends through the rotatable mechanism to connect the fixing mechanism and the ultrasonic transducer.

3. The device according to claim 2, wherein the cable is a flexible cable and wherein the cable connects the fixing mechanism and the ultrasonic transducer through a center of the rotatable mechanism.

4. The device according to claim 1, wherein the ultrasonic transducer comprises a plurality of ultrasonic transducers having different shapes and uses.

5. The device according to claim 1, wherein the ultrasonic transducer is connected to the center of the rotatable mechanism through a center of the ultrasonic transducer, or through a position other than the center of the ultrasonic transducer.

6. The device according to claim 4, further comprising a detection switch or a position sensor configured to detect a change of position of the ultrasonic transducers of different uses and to allow the related ultrasonic transducer to work.

7. The device according to claim 1, wherein the ultrasonic transducer is a multifunctional ultrasonic transducer or a combination of a plurality of mono-functional ultrasonic transducers.

8. The device according to claim 1, further comprising a locking mechanism for locking the ultrasonic transducer at a predetermined position.

9. A detection method using an ultrasonic probe device, the method comprising:
connecting an ultrasonic transducer to a fixing mechanism through a rotatable mechanism so that the ultrasonic transducer is rotatable on the fixing mechanism; and
rotating the ultrasonic transducer to a predetermined position for scanning.

10. The method according to claim 9, further comprising:
providing a cable assembly comprising a cable and a connector, wherein the cable extends through the rotatable mechanism to connect the fixing mechanism and the ultrasonic transducer.

11. The method according to claim 10, wherein the cable is a flexible cable, and wherein the cable connects the fixing mechanism and the ultrasonic transducer through a center of the rotatable mechanism.

12. The method according to claim 9, wherein rotating the ultrasonic transducer to a predetermined position for scanning comprises:
determining whether the ultrasonic transducer has rotated to the predetermined position;
locking the ultrasonic transducer at the predetermined position upon determining that the ultrasonic transducer has rotated to the predetermined position; and
using the ultrasonic transducer for scanning.
